(19) United States
(12) Patent Application Publication
(10) Pub. No.: US 2011/0130560 A1
(43) Pub. Date: Jun. 2, 2011

(54) SONICATION CARTRIDGE FOR NUCLEIC ACID EXTRACTION

(75) Inventors: Amir M. Sadri, Toronto (CA); Nenad Kircanski, Toronto (CA); Manja Kircanski, Toronto (CA); Neven Nikolic, East Mississauga (CA); Milija Timotijevic, Toronto (CA)

(73) Assignee: BIO-RAD LABORATORIES, INC., Hercules, CA (US)

(21) Appl. No.: 12/788,777

(22) Filed: May 27, 2010

Related U.S. Application Data
(60) Provisional application No. 61/182,183, filed on May 29, 2009.

(51) Int. Cl. C07H 1/00; B01F 11/02

(52) U.S. Cl. 536/55.3; 366/110

(57) ABSTRACT

A cartridge in which sonication is applied to biological matter to disrupt and release nucleic acids from the matter is formed from a cartridge body containing a series of wells connected by fluid passages engineered to prevent backflow, with at least one well containing a sonication window covered by a thin lamina to transmit sonic vibrations from a sonication horn contacting the exterior surface of the window. Fluid transport among the wells is achieved by pressure differentials through the fluid passages, and a succession of functions is performed in the various wells, including disruption, mixing, binding of the released nucleic acids to binding materials, washing, elution, and collection.

### CROSS-REFERENCE TO RELATED APPLICATION

This application claims the benefit of U.S. Provisional Patent Application No. 61/182,183, filed May 29, 2009, the contents of which are incorporated herein by reference.

### BACKGROUND OF THE INVENTION

1. Field of the Invention

This invention resides in the field of nucleic acid extraction from biological cells and from soft and hard biological tissue.

2. Description of the Prior Art

The extraction of nucleic acids from tissue, fungi, bacteria and other cellular matter, as well as non-cellular structures such as viruses, is used in a wide variety of procedures in molecular biology and biomedical diagnostics, serving useful applications in both research and medicine. The extraction methods include both chemical and physical methods, each with their own advantages and each with limitations. Chemical methods tend to be easier to control and to provide more uniform and consistent results, while physical methods avoid the use of harsh chemicals. One physical method is sonication, and procedures have been developed using a sonication horn in direct contact of cells or a cell suspension, while others use indirect contact, such as through the wall of a sample container. In both the direct and indirect methods, beads with diameters of 250 microns or less are typically mixed in with the sample to enhance the sonication effect. Nevertheless, sample manipulation, extraction efficiency, and the avoidance of contamination remain goals that are difficult to achieve.

### SUMMARY OF THE INVENTION

The present invention resides in a cartridge for nucleic acid extraction by sonication with the use of an external sonication horn, and in methods for nucleic acid extraction from biological cells, soft tissue, hard tissue, and biological matter in general by use of the cartridge. Sonication of the cells or tissue is thus achieved without direct contact between the sonication horn and the sample, and lysis of the cells or tissue is preferably achieved without the use of beads or any solid material in direct contact with the sample, other than the walls of the cartridge itself. Sonication occurs in a sample well to which sonic vibrations are transmitted through a sonication window in the wall of the well which is also a side wall of the cartridge, with the assistance of variable pressure, preferably an oscillating pressure at a subsonic frequency, in the sample well to agitate the well contents and enhance the disruption of the biological matter. The sonication window is covered with a lamina, or generally any thin layer or membrane, of a material that is deflectable by sonic vibrations, and the creation of sonic vibrations in the sample well is achieved by vibrating the horn while the horn is close to or in contact with the outer surface of the lamina. In preferred embodiments, as explained further below, cell or tissue disruption can be promoted by one or more enhancements to simple sonication in addition to the variable pressure. These include the use of ultrasonic vibrations applied in pulses, and using a sample well that is shaped to cause the sample to circulate within the well as vibrations are applied or between pulses.

The sample well is one of a series of wells in which a succession of functions is performed with the result of obtaining the extracted nucleic acid in an isolated and purified form, in high yield, and at a rapid rate, and the cartridge contains fluid passages between the various wells that are configured to prevent back flow by including a vertical connecting channel arranged such that the fluid enters the channel at the bottom and leaves at the top. The term “vertical” is used herein to denote a direction with a vertical component. Channels in which the vertical connecting channel is itself vertical (i.e., perpendicular to the upper surface of the cartridge) are preferred. Thus, a liquid from one well is drawn, or otherwise caused to flow, from the bottom of the well into the bottom of the vertical channel, then up the channel, and finally from the top of the channel into the top of the receiving well. Since each well typically contains a head space occupied by air or an inert gas, above the liquid level, momentary reversals of pressure drops between wells will not result in liquid entering the fluid passage opening at the top of the well. In addition to functional wells in which the sample or lysis products are treated or collected, the cartridge contains, in preferred embodiments of the invention, one or more additional wells serving as buffer reservoirs and one or more pressure/vacuum ports through which pneumatic pressure or a partial vacuum is applied to individual wells for purposes of conveying the fluids through the fluid passages and into, out of or between the various wells. With the use of these channels in conjunction with the application of controlled vacuum or pressure on the ports that lead to the various wells, cartridges in accordance with the present invention avoid the need for valves incorporated in the cartridges themselves. The elimination of internal valves allows the cartridges of this invention to be manufactured at less cost than cartridges that contain such valves.

The cartridge also permits the user to select an extraction protocol and to adapt the protocol to the specific needs of the sample, by varying the types and quantities of the various buffers and wash liquids used and the degree and level of agitation for purposes of optimizing yield and uniformity. The cartridge is used in conjunction with a manifold which provides buffer solutions to individual wells and imposes the pressure differentials through the pressure/vacuum ports that are used to transport the fluids between different parts of the cartridge.

These and other objects, features, and advantages of the invention will be more apparent from the attached Figures and the description that follows. The term “nucleic acid-containing biological matter” is used herein for convenience to include any biological structure that encapsulates or otherwise retains a nucleic acid that a researcher or a clinician seeks to extract, and from which the nucleic acid can be released by sonication.

### BRIEF DESCRIPTION OF THE DRAWINGS

FIG. 1 is a perspective view of a sonication cartridge in accordance with the present invention.

FIG. 2 is a horizontal cross section of a sample well of alternative shape to the sample well of the cartridge of FIG. 1.

FIG. 3 is a vertical cross section of the cartridge of FIG. 1 taken along the line 3-3 of FIG. 1.

FIG. 4 is a vertical cross section of the cartridge of FIG. 1 taken along the line 4-4 of FIG. 1.

FIG. 5 is a perspective view of a series of cartridges in accordance with the present invention supported on a rack with a sonication horn arranged for sonication of samples within the cartridges.

### DETAILED DESCRIPTION OF THE INVENTION AND PREFERRED EMBODIMENTS

Among the wells in the cartridge in preferred embodiments of the present invention are:
a sample (sonication) well in which the sample is initially placed and disruption of the nucleic acid-retaining matter occurs, the well optionally containing a mesh filter to impede the passage of particles greater than a preselected diameter from the well (the cut-off diameter will vary according to the needs of the particular sample or system; in some cases it may be 20 microns, for example, in others 10 microns, in others 1 micron, and in others 0.22 microns),
a mixing well in which the lyses can be further treated prior to nucleic acid recovery, such as with additives and further suspending agents for various purposes,
a binding well that retains a solid binding material that binds selectively nucleic acids in preference to other lysis components such as proteins and tissue or cell wall fragments,
a species extract collection well in which the nucleic acids extracted in the binding well can be collected and retained for study, or a vial that is easily detached from the cartridge and serves the same purpose, and
a waste collection well in which components of the sample remaining after the extraction can be deposited.

The sonication window in the external wall of the sample well provides sonication access to the sample. In the sample well, the sample suspended in the lysis buffer is exposed to disruptive forces causing rupturing of sample tissue matrix, cell membranes and other intra-cellular objects allowing nucleic acids to be released to the liquid. As noted above, the disruption can be promoted by one or more enhancements. One of these enhancements is the use of pulsed ultrasonic waves for the sonication. Another enhancement is the pressurization of the sample well contents with a variable pressure to promote the sample disruption and the movement of liquid within the well, and also to reduce the occurrences of sonication “blind spots,” i.e., sites within the well at which the sonication wave intensity is lower than the target intensity. A still further enhancement is the use of a sample well with a convex reflection wall, i.e., the wall opposite the wall in which the sonication window resides. A convex reflection wall can enhance the natural circulation of the liquid within the sample well.

A further feature that appears in certain embodiments of the cartridges of this invention is a second sonication window in an external wall of the mixing well to allow sonication to be used in the mixing stage. An alternative to sonication in the mixing well is the bubbling of air or inert gas through the well. Such bubbling can be produced by applying slightly positive air (or inert gas) pressure on one of the air ports. Increased pressure through the air port to the binding well, for example, can cause air bubbles to form in the mixing well at the mouth of the channel that connects the mixing and the binding well. Other alternatives will be readily apparent to those experienced in the processing of cell lysates. One such additional alternative is the application of a varying pressure, such as an oscillating pressure at a frequency below sonic frequencies, to a wall of the well through a flexible membrane in the wall or through one of the ports that supply pressurized air (or inert gas) or vacuum. Agitation by pressure oscillations can be used on both the mixing well and the sample (sonication) well, in which case the pressure oscillations will be applied through a wall other than the wall through which sonic vibrations are transmitted.

The fluid passages include sample transfer passages that join the various wells. One sample transfer passage will lead from the sample well to the mixing well, another from the mixing well to the binding well, still another from the binding well to the species extract collection well, and still another from the binding well to the waste collection well. The timing, sequence, and coordination of the flows through these passages can be programmed or manually directed by the user through the aforementioned manifold. Cartridges in preferred embodiments will likewise contain buffer liquid ports in the top surface of the cartridge and fluid passages from these ports to various wells for the supply of buffer liquids to these wells, or buffer liquid reservoirs within the cartridge to contain the buffer solutions needed for the protocols, or both such ports and reservoirs. Pneumatic ports are also included in preferred embodiments to supply pressure or partial vacuum as mentioned above.

The cartridge can be formed of any of a variety of materials, including those that are commonly used in the construction of laboratory equipment. The body of the cartridge, i.e., the portion exclusive of the thin walls through which vibrations or pressure variations are transmitted, can for example be formed of polycarbonate or any other resin that is inert to biological fluids. A convenient method for forming the body is injection molding. The laminae forming the thin walls, termed “windows” in this specification, can be formed for example of polyester, polystyrene, or similar materials that are similarly capable of deflection upon contact with a sonication horn without rupture. A single lamina or two or more laminae can be used. The thickness of the laminae over the windows can vary widely, although for best results, laminae of thicknesses within the range of 50 to 200 microns, and preferably approximately 100 microns, are preferred. The window material and window size will be selected such that the natural vibration frequency of the window is substantially lower than the frequency of the sonic vibrations that are applied. The difference is preferably at least about 10 kHz, and most preferably at least about 20 kHz. As an example, sonic vibrations at a frequency of 30 kHz can be applied to a window made of material with a natural vibration frequency of 8 kHz.

Sonication, which term is used herein to include the use of ultrasound, can be achieved by conventional means through a sonication horn. A piezoceramic transducer for example can be used, and frequencies within the approximate range from about 25 kHz to about 40 kHz will most often be the most effective. Power levels can vary as well. It is presently contemplated that tissue and cell disruption in the sample cell be achieved with a sonication power level of approximately 10 watts. When sonication is used in the mixing well, a power level of approximately 5 watts will be sufficient to provide effective results. Sonication is preferably performed in pulsewise manner using a 60% to 80% duty cycle, for example 800 msec on and 200 msec off. The effect is further enhanced by overshooting the power at the beginning of each pulse. The duration of the sonication for disruption of a single sample will vary with the sample. For cells, for example, disruption can be achieved with 10 to 15 seconds of sonication, while for tissue, disruption can take from 1 to 2 minutes. Shorter periods of time can be used in the mixing well. Pulses can also be applied in multiple cycles with quiescent periods in between to allow cooling of the sample between each set of pulses. For either well, agitation of the well contents by pressure variations in addition to sonication can be achieved, for example, by varying air pressure through a port connected to the well while keeping all other air ports closed. Variable pressure can also be applied through a flexible membrane other than the sonication window, using a servomotor or a peristaltic pump to cause the membrane to oscillate, for example at a rate of one to five oscillations per second.

For most effective results in applying the sonic vibrations, the sonication horn is preferably maintained at a predetermined distance from the sonication window lamina. The optimal distance is readily determinable by routine testing and is preferably maintained for all cartridges when a series of cartridges is sonicated in succession. When the cartridges are mounted on a rack, for example, the distance can be maintained by appropriate spacing members on the rack or on the moving part carrying the sonication horn. The moving part, for example, advance the sonication horn tip to a fixed offset from the sonication window, the offset being the same for all cartridges on the rack.

While the invention is capable of a wide range of constructions and implementations, its features are best understood by an examination of a specific example. One such example is shown in the Figures and described below.

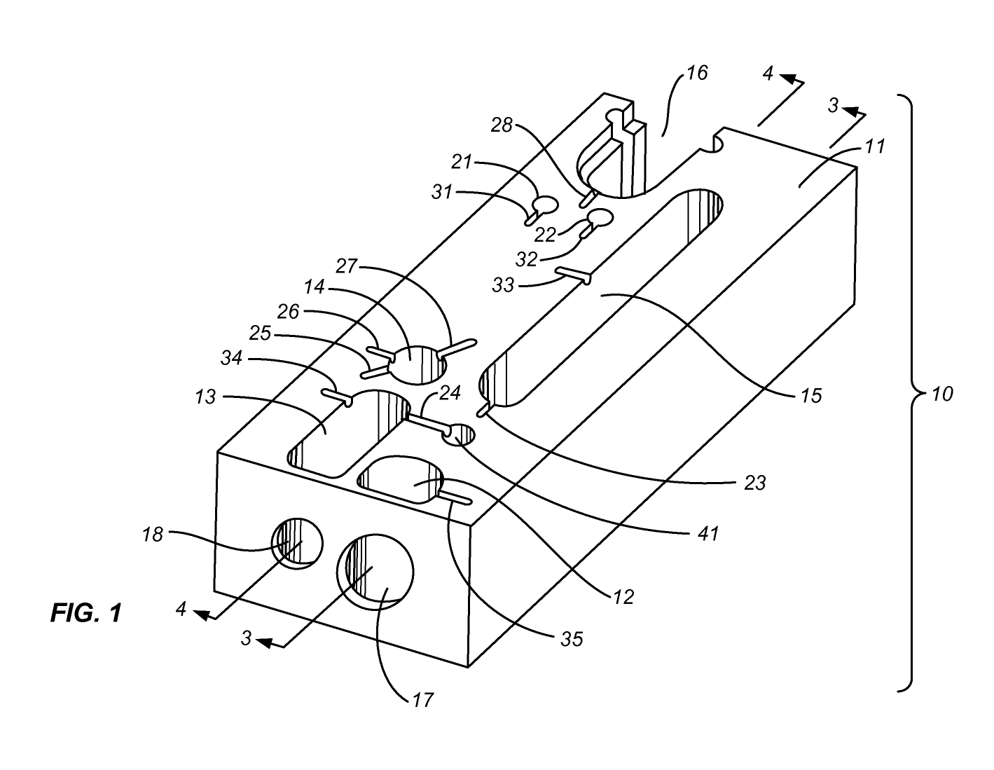

FIG. 1 shows the body 10 of a cartridge in accordance with this invention in a perspective view, with the upper and lower laminae removed to show the various wells, fluid passages connecting the wells, windows for the ultrasonic horn, and access ports for liquid buffers and for pressurized air or vacuum to move the fluids. The parts of the cartridge are described herein in reference to a reference plane, which is parallel to the top surface 11 of the cartridge body in the orientation shown in FIG. 1, with the wells distributed along the reference plane. In use, the cartridge is oriented with the reference plane horizontal, as shown in FIG. 1, and descriptions herein that refer to the tops and bottoms of the wells, to the vertical channels, and to the tops and bottoms of the vertical channels, are all made in reference to the horizontal orientation of the reference plane.

The laminae if shown would close the tops and bottoms of the wells, the windows that the sonication horn contacts for transmission of its vibrations to the well interiors, and some of the fluid passages. The wells include a sample well 12, a mixing well 13, a binding well 14, a waste collection well 15, and a species extract (i.e., nucleic acid) collection well 16. The species extract collection well 16 is depicted as a recess to receive a microtube in which the extract can be collected and removed for analysis. The windows 17, 18 for the sonication horn are located at the forward end of the cartridge body. The lamina that covers both windows when the cartridge is in use is flexible to allow transmission of the sonic vibrations. One window 17 communicates with the interior of the sample well 12 while the other window 18 communicates with the interior of the mixing well 13.

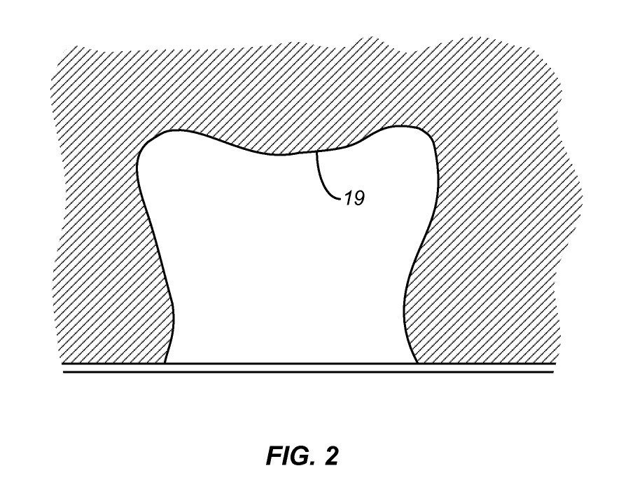

While the sample well 12 of the cartridge of FIG. 1 has a cross section with a concave back wall opposite the sonication window 17, a sample well of an alternative cross section is shown in FIG. 2. The back wall 19 of this well is convex rather than concave, and by virtue of its convex contour this wall causes the sonic vibrations to be distributed more effectively through the well. The convex back wall acts as a convex reflective mirror for the waves induced by the oscillating membrane. In this particular embodiment, the waves split in two main vortices to distribute the exposure of the tissue sample to the sonic vibrations. Back walls of other shapes can be used to produce a different number and distribution of vortices to achieve optimum performance for different samples or for sample wells of different sizes.

Returning to FIG. 1, additional wells 21, 22 are used as supply reservoirs for wash buffers. The fluid passages that provide transport of the various fluids between the wells each include vertical channels (not visible in this view) extending the full height of the cartridge body 11 and short horizontal upper and lower grooves at the top and bottom, respectively, of each vertical channel to connect the vertical channels with the wells. The upper connecting grooves 23, 24, 25, 26, 27, 28 are visible in FIG. 1. In each case, fluid is drawn from the bottom of a well into a lower connecting groove, then upward through a vertical channel, across through an upper connecting groove, and into the succeeding receiving well. The driving force is typically a vacuum applied to the receiving well or to a well downstream of the receiving well by additional connecting passages. Alternatively, the driving force can be produced by applying positive pressure on the well containing liquid (input or source well) relative to the pressure in the receiving well, which will typically be atmospheric pressure. With this arrangement of vertical channels and horizontal connecting grooves, fluid is inhibited from flowing backwards through a fluid passage and contaminating wells that are upstream in the well sequence. The pressure and vacuum access ports are additional grooves 31, 32, 33, 34, 35 in the top of the cartridge body 11, drawing vacuums on, or applying pressure to, individual wells, or for supplying fluids from outside the cartridge. These ports and grooves can also serve an additional function, specifically agitation of the well contents by the intermittent application of high pressure. The groove 35 leading to the sample well, for example, can be used for applying a varying pressure pulses to the contents of the well to supplement the sonication and thereby assist in the release of nucleic acids from the sample, particularly when the sample consists of tissue.

In a typical protocol, a liquid sample in which the nucleic acid-containing biological matter is suspended is placed in the sample well 12, and the sonication horn is brought in contact with the sonication window 17 of the sample well. Sonication is performed at a sufficient intensity and duration to disrupt the biological matter in the sample, and a vacuum is then applied to the mixing well 13 through the vacuum access groove 34 which is joined to a manifold (not shown) at the top of the cartridge. The vacuum causes the filtrate from the disrupted matter, i.e., the fluid passing through the filter in the sample well, to pass through the fluid passage that includes a lower connecting groove (not visible) that leads to a vertical channel 41 and then to the upper connecting groove 24 to enter the mixing well 13. In the mixing well 13, ethanol from the manifold is added to the sample filtrate through an opening in the upper lamina. The sonication horn is then repositioned to the sonication window 18 of the mixing well and brief sonication is performed to mix the ethanol with the lyses in the filtrate to prevent the lyses from settling into two layers. As noted above, this brief sonication can be replaced by bubbling gas through the mixing well. In either case, the mixture of ethanol and lyses is then drawn into the binding well 14 by a similar application of vacuum that is drawn through the waste well 15, using a vacuum access groove 33 in the waste well, causing the mixture to enter the binding well 14 through a fluid passage 25. The binding well 14 contains a binding membrane that captures DNA, RNA, or both from the lyses, allowing the remainder of the fluid to enter the waste well 1 through a fluid passage 23 between the wells.

Before the nucleic acid is released from the binding membrane, the membrane is washed to purify the retained nucleic acid. This washing can be performed by wash buffers, and both a low stringency buffer and a high stringency buffer are stored for this purpose in separate wells 21, 22 of the cartridge, each of these wells communicating with the binding well 14 through separate fluid passages. Individual movement of the two buffers to the binding well is achieved by individual pressure ports 31, 32. Once washing is complete, release of the nucleic acid from the binding membrane is achieved by the use of an appropriate elution buffer suited to detach (elute) nucleic acid from the binding membrane. The elution buffer with the nucleic acid dissolved therein is then drawn into the collection well 16, where a thermoelectric element maintains the solution temperature at 0-10° C. An alternative construction of the cartridge is one that includes an auxiliary well between the binding well and the collection vial, with a thin lamina on the bottom of the auxiliary well and the thermoelectric element in contact with the outer surface of the lamina. Effective cooling can be achieved with an auxiliary well that is relatively small (one that is but a few mm in diameter, for example) and accordingly a small and inexpensive thermo element.

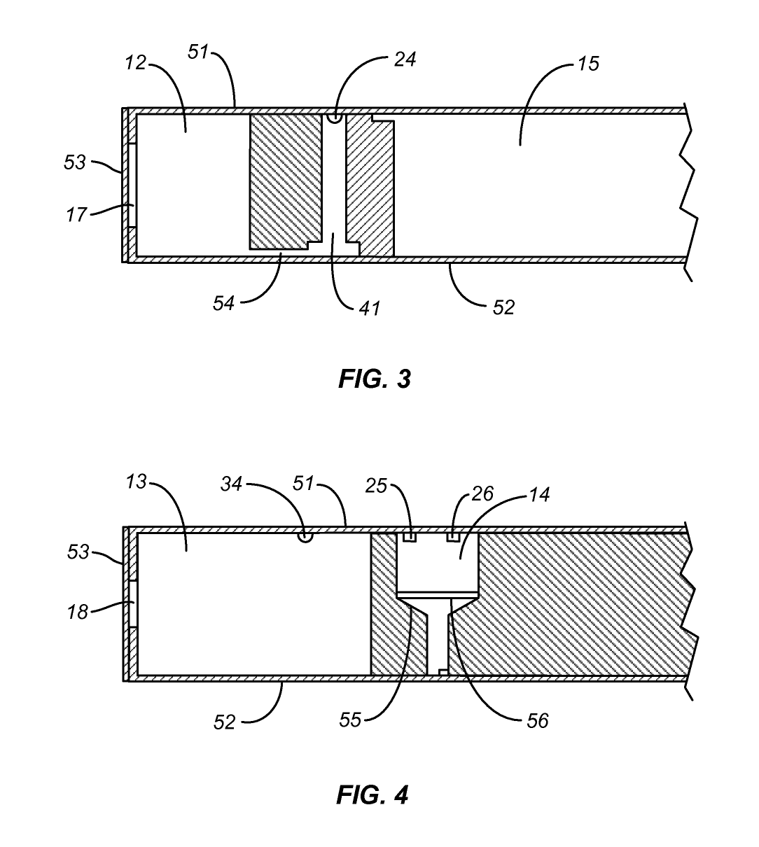

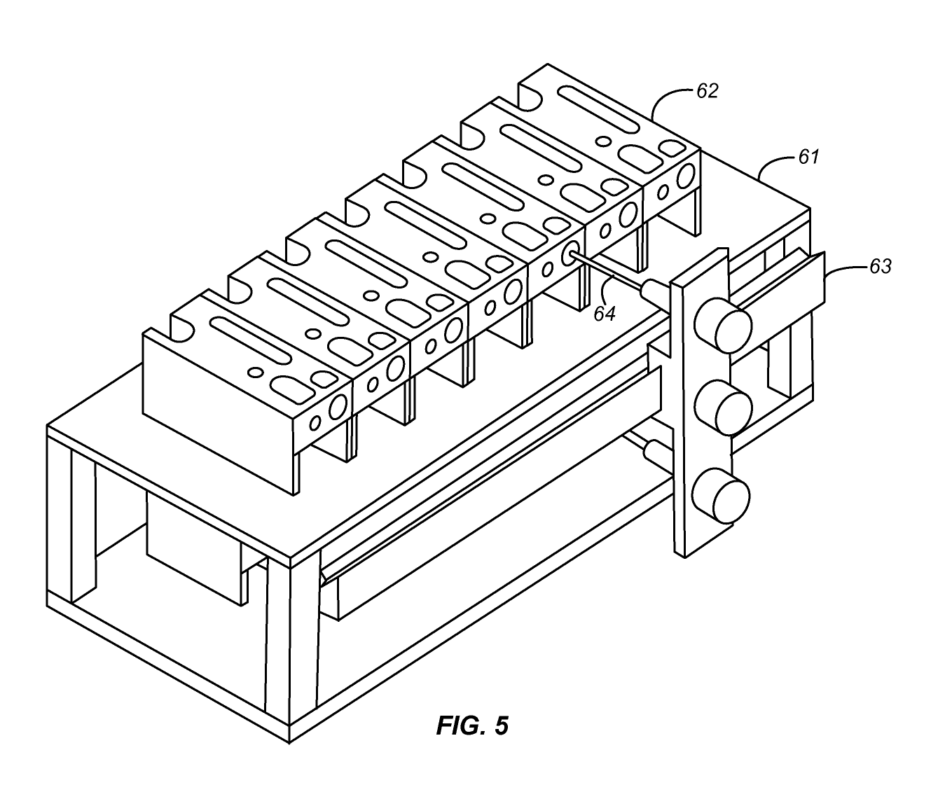

As noted above, the fluid passages between the wells consist of horizontal grooves, which become closed channels when covered with the laminae at the top and bottom of the cartridge body, joined by vertical channels in an arrangement designed to prevent backflow of the various fluids which might contaminate the fluids in the upstream wells. The passages are oriented in various directions depending on which wells they are designed to connect and the particular direction of flow they are intended to allow or prevent. One such passage is shown in FIG. 3, which is a cross section of the front end of the cartridge body taken along the line 3-3 of FIG. 1. This cross section shows the sample well 12 and the waste well 15, as well as the sonication window 17 at the forward end of the sample well 12. A parallel cross section is shown in FIG. 4, taken along the line 4-4 of FIG. 1 to show the mixing well 13 and the binding well 14. FIG. 3 and FIG. 4 also show the laminae that are not shown in FIG. 1. These laminae include an upper lamina 51, a lower lamina 52, and a front end lamina 53, the front end lamina 53 covering both the sonication window 17 on the sample well and the sonication window 18 on the mixing well, but thin enough (100-200 microns, for example) to transmit sonic vibrations through either window. The fluid passage shown in FIG. 2 is one that connects the sample well 12 (FIG. 2) with the mixing well 13 (FIG. 3), and includes, in the direction of flow, a lower horizontal connecting channel 54 at the level of the floor of the sample well 12, the vertical channel 41 (also shown in FIG. 1), and an upper horizontal connecting channel formed from the horizontal groove 24 that leads to the mixing well and is shown in FIG. 1. The upper horizontal connecting channel in this case is at a right angle to the lower horizontal connecting channel 54. Since fluid from the mixing well can only enter the vertical channel 24 through the upper channel 24 at the top of the mixing well, backflow from the mixing well to the sample well is thus prevented. The same arrangement prevents backflow from all wells.

FIG. 4 also shows that the profile of the binding well 14 includes a tapered middle section 55 which supports the binding membrane 56. The direction of flow through the binding well 14 is down, through the binding membrane 56 and out of the well by way of a flow passage that begins with a horizontal channel 57 at the level of the binding well floor.

The upper lamina 51 serves a function in addition to that of sealing the tops of the wells and the fluid passages. This function is the supplemental mixing function discussed above, by to flexure of the lamina to agitate the contents of the underlying well(s). The flexure can be induced by any conventional means of applying a variable force. One such means is a peristaltic pump that is placed in direct contact with the lamina.

A support rack 61 for holding several cartridges is shown in FIG. 5. The cartridges 62 are mounted on the rack in a linear arrangement and the rack includes a track 63 along which a sonication horn 64 can be conveyed, causing the horn to engage each of the cartridges in succession. The rack shown holds two rows of seven cartridges each, and supports two sonication horns, one for each row. Other arrangements of rows of cartridges, columns or both, and other rack sizes can likewise be used. To sonicate the wells in succession, the sonication horn(s) can be mounted to a motorized stage to carry the horn(s) from one cartridge to the next and to advance the horn to the desired distance from the sonication window lamina.

Variations on the cartridges shown in the drawings will be readily apparent to those of skill in the art. Certain wells can be eliminated or combined. Additional wells can be included, such as an enzyme well containing RNase or DNase joined to the binding well through corresponding channels of the same configuration as those shown. Another variation is the inclusion of an auxiliary collection well described above for higher cooling efficiency. Thermoelectric elements can be included at various locations, particularly along the lower surface of the cartridge, for cooling of the well contents, particularly the species extract well and the enzyme wells in cartridges that contain enzyme wells. The protocols can also vary. Abrupt or continuous changes of pressure (including vacuums) can be applied to particular ports to cause liquid to flow from one well to another through the interconnecting channel network. A continuous change in pressure is particularly useful in minimizing transient effects that may otherwise cause liquid to flow in an unintended direction. The protocol can run in either a batch mode or a continuous mode. Batchwise transfers of liquid are particularly useful when transferring liquid from one well to a smaller well. Excess liquid can then be directed to the waste collection well by drawing a vacuum on the waste collection well.

In the claim or claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A cartridge for extracting nucleic acid from nucleic acid-containing biological matter, said cartridge comprising a cartridge body having a reference plane and a top surface parallel to said reference plane, said cartridge body comprising a plurality of wells distributed along said reference plane, said wells connected by a network of sample transfer passages oriented such that, when said reference plane is horizontal, each sample transfer passage comprises a channel extending from the bottom of one well to the top of a succeeding well through a vertical connecting channel, and said plurality of wells comprising a sample well and a sonication window opening into said sample well through a side wall of said cartridge body, said sonication window covered by a lamina of material deflectable by sonic vibrations generated by a sonication horn, said sample well further comprising means for applying a variable pressure to said sample well to agitate well contents during sonication.

2. The cartridge of claim 1 wherein said lamina covering said sonication window has a natural vibration frequency substantially below sonic.

3. The cartridge of claim 1 wherein said plurality of wells further comprises a binding well with a solid binding material therein that binds nucleic acids.

4. The cartridge of claim 3 wherein said plurality of wells further comprises a mixing well and means for mixing liquid in said mixing well, and said network of sample transfer passages comprises a first sample transfer passage leading from said sample well to said mixing well, and a second sample transfer passage leading from said mixing well to said binding well.

5. The cartridge of claim 4 wherein said plurality of wells further comprises a waste collection well and a species extract collection well, and said network of fluid sample passages further comprises a third sample transfer passage leading from said binding well to said waste collection well and a fourth sample transfer passage leading from said binding well to said species extract collection well.

6. The cartridge of claim 1 further comprising a plurality of buffer liquid ports at said top surface, each buffer liquid port communicating with a well through a buffer passage comprising a vertical channel extending from said buffer liquid port and a horizontal channel extending from said vertical channel to an opening in the bottom of said well.

7. The cartridge of claim 1 further comprising a plurality of buffer liquid reservoirs, each buffer liquid reservoir to a well by a buffer reservoir passage oriented such that, when said reference plane is horizontal, each buffer reservoir passage comprises a vertical channel extending from said buffer liquid reservoir and a horizontal channel extending from said vertical channel to an opening in the bottom of said well.

8. The cartridge of claim 1 further comprising pneumatic ports at said top surface, joined to said wells through connecting passages to impose pressure differentials between wells to cause fluid to flow between wells through said sample transfer passages or to apply intermittent pulses of elevated pressure to agitate the contents of a well.

9. The cartridge of claim 1 wherein said sample well has a vibration reflecting wall opposite said sonication window, said vibration reflecting wall being shaped to induce multiple vortices of fluid movement in response to sonic vibrations introduced through said sonication window.

10. The cartridge of claim 1 further comprising a filter in said sample well to impede passage of particles greater than a preselected diameter.

11. A method for extracting nucleic acid from nucleic acid-containing biological matter, said method comprising:
(a) placing a suspension of said nucleic acid-containing biological matter in a sample well of a sonication cartridge comprising a cartridge body having a reference plane with top and bottom surfaces parallel to said reference plane, said sample well being one of a plurality of wells in said cartridge body distributed along said reference plane, said wells further comprising a binding well with a solid binding material therein that binds nucleic acids, a waste collection well, and a species extract collection well, said plurality of wells connected by a network of sample transfer passages oriented such that, when said reference plane is horizontal, each sample transfer passage comprises a channel extending from the bottom of one well to the top of a succeeding well through a vertical connecting channel, said cartridge body further having a sonication window opening into said sample well through a side wall of said cartridge body, said sonication window covered by a lamina of material deflectable by a sonication horn;
(b) applying sonication energy to said suspension through said lamina covering said sonication window to convert said suspension to a lysate, and applying variable pressure to said sample well at a subsonic frequency to agitate said suspension;
(c) conveying said lysate through a first sample transfer passage into said binding well under conditions causing nucleic acids in said cell lysate to bind to said solid binding material, and expelling unbound components of said cell lysate through a second sample transfer passage into said waste collection well;
(d) contacting said nucleic acids so bound with an elution buffer having a nuclease suspended therein to release said nucleic acids into said elution buffer; and
(e) conveying said released nucleic acids through a third sample passage into said species extract collection well.

12. The method of claim 11 wherein step (b) comprises contacting said lamina with a sonication horn and vibrating said sonication horn while so contacted at a sonic frequency.

13. The method of claim 11 wherein said plurality of wells further comprises a mixing well and step (c) comprises conveying said cell lysate first to said mixing well through a fourth sample transfer channel and agitating said cell lysate in said mixing well, then conveying said cell lysate to said binding well through said fits sample transfer channel.

14. The method of claim 11 wherein said conveying steps are performed by applying pressure differentials through said sample transfer passages.

15. The method of claim 14 wherein said pressure differentials are produced by application of pressurized air or inert gas.

16. The method of claim 11 wherein said nucleic acid-containing biological matter is biological cells.

17. The method of claim 11 wherein said nucleic acid-containing biological matter is hard or soft biological tissue.
